(19) United States
(12) Patent Application Publication
(10) Pub. No.: US 2024/0002898 A1
(43) Pub. Date: Jan. 4, 2024

(54) BIOCOMPATIBLE COMPOSITE MEMBRANE, METHOD FOR FABRICATING THE MEMBRANE, BIOREACTOR AND METHOD FOR INVESTIGATING CELLS ATTACHED TO THE BIOCOMPATIBLE COMPOSITE MEMBRANE

(71) Applicants: Helmholtz Zentrum Muenchen - Deutsches Forschungszentrum Fuer Gesundheit Und Umwelt (GmbH), Neuherberg (DE); Julius-Maximilians-Universitaet Wuerzburg, Wuerzburg (DE)

(72) Inventors: Ali DORYAB, Olching (DE); Otmar SCHMID, Muenchen (DE); Andreas SCHROEPPEL, Unterschleissheim (DE); Juergen GROLL, Eibelstadt (DE); Mehmet Berat TASKIN, Wuerzburg (DE)

(21) Appl. No.: 18/038,393

(22) PCT Filed: Nov. 24, 2021
(86) PCT No.: PCT/EP2021/082887

§ 371 Date: May 23, 2023

(30) Foreign Application Priority Data
Nov. 24, 2020 (LU) LU102222

(51) Int. Cl. C12Q 1/02; C12M 1/12; C12M 1/34; C12M 1/00

(52) U.S. Cl. CPC C12Q 1/02; C12M 25/04; C12M 41/40; C12M 29/10; C12M 41/46

(57) ABSTRACT

In various embodiments a biocompatible composite membrane for in vitro cell culturing comprising a first material, which is non-water soluble and a water soluble second material is provided, wherein the composite membrane comprises a porous scaffold and a filling layer, the scaffold comprising the first material and the filling layer comprising the second material. Further, a method for fabricating the membrane, a bioreactor for use of the membrane in cell-stretch experiments and a corresponding method for investigating cells attached to the biocompatible composite membrane are also provided.

The present invention relates to a biocompatible composite membrane, method for fabricating the membrane, a bioreactor and method for investigating cells attached to the biocompatible composite membrane, preferably under air liquid interface (ALI) culture conditions

In vitro organ models are intended to at least partially replace animal experiments. Following Albert Einstein's premise, in vitro models have to be as complex as necessary to be predictive for clinical outcome, but not more complex than that. Albeit aerosol inhalation is widely used for treatment of lung diseases, more than 95% of preclinical in vitro testing related to the development of drugs delivered to the lung is still carried out with standard submerged cell cultures, where the cells are seeded on the bottom of (plastic) wells where they are completely covered with a cell culture medium. Such an experimental cell condition is referred to as a submerged cell culture condition. This condition is non-physiological from the perspective of inhalation therapy or inhalation toxicology since, in contrast to the conditions in a real lung, aerosolized drugs or toxins are not deposited directly onto the lung epithelium. This can now be accomplished with so-called air liquid interface (ALI) cell culture systems, where cells are seeded on the apical (air) side of a porous/perforated membrane, which is in contact with cell culture medium located on the opposite site of the membrane, the basal side. Such a setup provides for more physiological aerosolized drug/toxin delivery and polarized cell culture conditions resembling a realistic air-blood barrier and is therefore more biomimetic than submerged culture conditions.

It has been recognized that, in addition to ALI conditions, elasticity of the membrane, which the cells are cultured on, and breathing-induced cyclic cell-stretch as a stimulus are also important aspects relating to the environment of lung cells and have a significant impact on cellular response under in vitro conditions. Hence, there is currently significant effort to combine ALI with cell-stretch conditions in bioreactors which are to mimic conditions prevalent in a lung, including lung-on-a-chip technologies.

One of the main limitations of all present in vitro cell culture systems of the lung relates to the nature of the membrane used for cell seeding/growth. The main problem when designing suitable membranes for cell-stretch cell experiments under ALI conditions arises from a natural incompatibility of the membrane characteristics which are most relevant during each one of the two phases of ALI cell culture experiments.

In the first phase, initial cell adhesion and growth requires a non- or at least low-porous, wettable membrane which is conducive to cell growth and formation of a confluent cell layer on the air-facing side of the membrane. This requirement governs the experimental setup for the first few days until a cell culture in the form of a single confluent cell layer on the air-facing side of the membrane (here: first surface) has been cultivated. Co-culturing with other cells both on the first and/or the second (medium-facing) surface is possible.

In the second phase, i.e. once one or more confluent cell layers have been formed on the surfaces of the membrane, drug/toxicity testing experiments are performed under ALI and stretch conditions requiring a porous (for cell-medium contact across the membrane), elastic, stretchable, durable and biocompatible membrane. As can be seen, those properties of the membrane are in many aspects the opposite of the properties of the membrane which are desirable for the first phase.

From the perspective of the cells the main problem is that elastic materials that are typically used for the elastic membranes are not conducive to cell growth and, vice versa, materials that are conducive to cell growth are not elastic and/or not durable enough for use in cell culture medium. All of the currently available membranes are problematic in this respect and thus impose severe limitations on the underlying experimental setup.

Another problem is the lack of technology for real-time monitoring/controlling of both the strain applied to the cells and membrane elasticity during cell stretch experiments under ALI conditions. This drawback is particularly relevant since the mechanical properties of the elastic cell-covered membrane may vary during the course of an experiment. This variance, which may take place undetected, would alter the elasticity of the membrane and thus the strain conditions which may possibly lead to an altered cellular response. Moreover, an increase of stiffness (decrease of elasticity) of cells is one of the hallmarks of various lung diseases, such as fibrosis. Hence, elasticity monitoring can serve as real-time diagnostic tool for pathophysiologic variations, which is essential for drug efficacy testing.

Currently, two approaches are known which both aim at overcoming the technical hurdles arising from the fact that stretchable materials are typically not conducive to cell growth and vice versa.

According to a first approach, a membrane comprising a stretchable material is coated with extracellular matrix (ECM) proteins, such as collagen, fibronectin or Matrigel, which is a commercial product, immediately prior to cell seeding to make the membrane conducive to cell growth.

The membrane itself can be fabricated with various types of methods, for instance with spin coating of stretchable materials (mainly hydrophobic silicon-based membrane materials such as PDMS (polydimethylsiloxane) or Sylgard 184). Once fabricated, the hydrophobic membrane is made conducive to cell growth by coating it with ECM proteins immediately prior to cell seeding. Silicone-based materials are elastic and provide good gas permeability, optical transparency, low cost, chemically inert and easy to use. However, these membranes are limited in terms of porosity requiring pore sizes of less than approx. 3 μm to avoid cells from migrating through the membrane during the initial growth phase and porosity (<10%) to provide sufficient structural integrity for ultra-thin (<10 μm thick) membranes during stretch experiments. This may lead to dislodging of cells during stretch experiments.

Alternatively, stretchable membranes may be fabricated with electrospinning of polymers which results in a finely woven membrane consisting of nano-sized threads consisting of a polymeric material. Such electrospun membranes are made of e.g. polycarbonate polyurethane (PCU), polycaprolactone (PCL), polydioxanone (PDS) and poly(lactic-co-glycolic acid) (PLGA).

Membranes fabricated utilizing that approach typically provide acceptable results in terms of stretch conditions. However, the membranes are typically subject to limitations regarding cell growth and proliferation due to the hydrophobic nature of these materials, which cannot be completely overcome by coating with ECM proteins. The use of natural proteins for enhanced conduciveness to cell growth also introduces experimental uncertainty since the ECM proteins have a significant batch-to-batch variability, just like most biological materials. In addition, the coating procedure involves additional expenses and requires time.

Furthermore, many of the stretchable materials adsorb (partially hydrophobic) molecules, which can alter protein/nutrient and drug concentration in the cell culture medium and hence affect cellular response and/or bias drug transport studies. Finally, some molecular components of silicon-based materials (e.g. PDMS) can leach into the cell culture medium, which may “poison” the cell culture medium with potentially adverse effects on cell biology.

Electrospun membranes may also bring about several disadvantages. The membranes fabricated in that manner are thicker than 20 μm (otherwise, they lack structural integrity), which is much larger than the basal membranes found in the lung which have a thickness in the order of approx. 100 nm. Furthermore, from a biological perspective ultra-thin electrospun membranes with nano-sized threads result in large pores (much larger than 3 μm) and large porosity (>20%) associated with low structural integrity making handling of the membrane difficult. Pores of this size (>3 μm) allow cells to migrate into the membrane rather than forcing them to form a tight epithelial monolayer directly at the ALI as found in the lung. Moreover, cells migrating into the membrane can form multi-layered cell cultures within the membrane matrix rather than a well-defined monolayer of cells at the surface of the membrane, i.e. directly at the ALI, as under physiological conditions. In any case, migration of the cells into the membrane matrix results in separation of the cells from air by some membrane material and does not reflect true ALI conditions.

In a second approach, co-polymers, i.e. polymers consisting of two or more polymeric materials, e.g. PCL & gelatin, can be used to generate relatively thick scaffolds (>70 μm thickness) for tissue engineering and regenerative medicine. These scaffolds are not stretchable and they are manufactured with different fabrication methods, such as electrospinning or freeze-drying. Furthermore, these membranes are not transparent enough for direct cell microscopy. In addition, some electrospun scaffolds and layer-by-layer bioprinting methods have been reported to mimic the basement membrane of alveolar epithelium and provide a better environment for cell proliferation with the thickness of the corresponding membranes lying in the range of 20-200 μm. From U.S. Pat. No. 8,647,837 a PET membrane with a coating of collagen and laminin is known for use in in vitro models of mammalian lung tissue. According to U.S. Pat. No. 10,350,795 flexible polymer films of poly(methyl methacrylate) (PMMA) can be formed, but they are not reported to be used for cell culture application.

Unfortunately, membranes based on co-polymers are often not as stretchable as lung tissue, which may adversely affect cell physiology and hence cellular response. Also, these membranes are often not transparent enough for direct cell microscopy. All of these artefacts are either detrimental to experimental simplicity, alter cell physiology towards a non-physiological state and/or reduce effective drug dose (e.g. muli-layered epithelial cell structures are formed within the membrane, which is non-physiologic and protects some of the epithelial cells from direct contact with the drugs). This is expected to mitigate reproducibility and relevance for prediction of clinical outcome, which is the main criteria for the relevance of in vitro cell models.

The aim of the present invention is to provide a membrane technology which solves at least some of the above-mentioned problems and, in particular, is able to provide a membrane which combines the seemingly contradicting properties required for the first phase (initial cell adhesion and cell proliferation) and the second phase (experiments, e.g. drug/toxicity testing) under stretch conditions in ALI experiments.

According to various embodiments, a membrane technology is provided which overcomes at least a vast majority of the above-mentioned deficiencies and offers additional benefits such as good prevention of cell migration into/across the membrane during the initial cell growth phase (no/low porosity) and high porosity and high elasticity during second phase, as well as gas permeability, optical transparency (for microscopy), low absorptivity of proteins from the cell culture medium, low cost and ease-of-use. Furthermore, in various embodiments a method for real-time online monitoring of the strain applied to the membrane and of the elastic modulus (as measure of elasticity) of the cell-covered membrane for well-controlled cell-stretch conditions and the cell layer itself is provided.

According to the present invention, a biphasic membrane is provided, which provides optimal properties for the two distinctly different phases of typical cell culture experiments mimicking “breathing” conditions, namely the initial first phase of cell growth followed by the second phase of cyclic stretch of a (confluent) cell layer under ALI conditions. This is accomplished by essentially two factors. First, a two-component (hybrid) polymeric material is used for manufacturing of the membrane, the material comprising, preferably consisting of gelatin and PCL (polycaprolactone), the former being chosen for its cell-conducive properties and the latter for its mechanical properties, in particular elasticity and structural integrity under highly porous conditions of ultra-thin membrane, respectively, as required for both the first phase and the second phase. Second, gelatin is used as a sacrificial material which is gradually dissolved when it comes in contact with the cell culture medium during the first phase of the experiments. By virtue of this process the originally non-porous and stiff membrane, which is very suitable for the first phase of the experiments, transitions into a porous/permeable and more elastic membrane, as required for the second phase of the experiments. As will be explained below in more detail, two variants of the biphasic membrane are presented herein. Independent of the variant, the membrane according to various embodiments may be transparent when it is configured for the second phase.

According to various embodiments, a biocompatible composite membrane is provided, comprising a non-water soluble, first material and a water soluble second material, wherein the composite membrane comprises a porous scaffold and a filling layer, the scaffold comprising the first material and the filling layer comprising the second material. The first material may be elastic, yet structurally resilient to stretch under ultra-thin (<10 μm), porous conditions.

In the context of the present application, a porous scaffold of the membrane relates to a scaffold in which the pores are open, pores which provide a connection between a first (e.g. upper) surface of the membrane and a second (e.g. bottom) surface of the membrane. In that sense, the pores of the membrane may be understood to correspond to “through-pores”. However, it is noted that the pore structure of the membrane may be such that not every opening on one side of the membrane is exclusively connected to an opening on the other side thereof. The structure of the membrane may be such that more than one opening on one side of the membrane may lead to more than one opening on the other side of the membrane. In other words, the membrane may comprise shared channels, such that almost every opening of a pore on one side of the membrane is connected to a (possible shared) opening on the other side thereof. Furthermore, the statement that the pores are through-pores is to be conceived that a substantial fraction or almost all pores are through-pores. However, due to the nature of the experimental process, some pores may be “blind pores”, i.e. pores which do not interconnect both surfaces of the membrane. However, those pores will typically correspond to a minority of all pores. In any case, the fraction of the blind pores is low enough to guarantee that the cells on the first side (apical side) of the membrane are connected to and nourished by the culture medium on the second side (basal side) of the membrane enough to remain vital under ALI conditions.

Furthermore, the porous scaffold of this embodiment is a fibrous scaffold, i.e. comprising fibers. According to various embodiments, the first material may be PCL and the second material may be gelatin.

According to further embodiments of the membrane, the porous scaffold may be obtained by electrospinning of a substance comprising the first material. In the following, the embodiments of the membrane comprising an electrospun membrane will be referred to as first embodiments of the membrane. Furthermore, the composite membrane according to the first embodiment may be obtained by spin coating of the second material onto the scaffold.

According to further first embodiments of the membrane, the porous scaffold may further comprise the second material.

According to further first embodiments of the membrane, the substance which is electrospun may comprise at least 5 to 15% % w/v, preferably 6 to 12% w/v, most preferably 7 to 10% w/v of the first material and 1 to 10% w/v, preferably 1 to 9% w/v, most preferably 2 to 8% w/v of the second material. According to yet further first embodiments of the membrane, the substance which is electrospun may only comprise the first material in the percentages of weight by volume as specified above. The compositions are given in w/v of the solvent, i.e. relating to the weight of the respective material in relation to the volume of a corresponding solvent. According to an exemplary second embodiment of the membrane, the first material and the second material may be dissolved in a solvent mixture for electrospinning at a weight ratio of the first material to the second material of 8:2. The solvent mixture may include 1,1,1,3,3,3-Hexafluoro-2-propanol (HFIP) and formic acid (FA) at a weight ratio of HFIP to FA of 9:1.

According to various embodiments, a method for fabricating of a biocompatible composite membrane, in particular the first embodiment of the membrane, is provided, comprising providing a porous scaffold by electrospinning of a substance comprising a first material, and providing a filling layer comprising a second material on the substrate. In the context of the first embodiments of the membrane, the filling layer may correspond to a covering layer as it predominantly covers the electrospun membrane scaffold. However, the covering layer may of course extend into void spaces contained in the electrospun membrane to some extent. The thickness of the covering layer may be smaller than the thickness of the electrospun scaffold.

The first embodiment of the membrane may be manufactured by electrospinning. The electrospinning process may be used to manufacture a biphasic ultrathin membrane. The membrane of this embodiment may have a thickness of 10 μm or less, preferably of 5 μm or less, more preferably 1 μm or less. During manufacture, the first material and the second material are mixed and electrospun into a fibrous and porous membrane. Subsequently, the fibrous membrane may be coated with a sacrificial covering layer of the second material, e.g. via spin coating, to facilitate initial cell adhesion and growth during the first phase. During the first phase, the second material is dissolved not only from the sacrificial layer, but also from the residual electrospun fibrous membrane, which turns the initially non-porous, non-elastic membrane into a porous, elastic membrane which then becomes well suitable for the second phase, i.e. the actual experimentation phase.

According to further embodiments of the fabrication method, it may further comprise the step of providing a desorption layer, preferably by spin coating, on a surface of a target onto which the substance is electrospun, the desorption layer preferably comprising the second material. The desorption layer may have a thickness of less than 500 nm, preferably less than 300 nm. The desorption layer acts as a spacer layer and facilitates the release of the electrospun membrane from the target surface of the electrospinning process such that it may be detached therefrom without any damage.

According to further embodiments of the composite membrane, it may comprise conglomerates of the second material which are embedded, as the filling layer, in the porous scaffold. In the following, those embodiments of the membrane will be referred to as second embodiments of the membrane. The conglomerates of the second material may correspond to “islands” of the second material, which is preferably a hydrophilic material, with the islands being formed during spin coating of the porous scaffold. The resulting filling layer may be comprised of a multitude of conglomerates or portions of the second material which may not necessarily need to be connected to one another. According to further embodiments of the composite membrane, the second embodiment of the membrane may be obtained by spin coating of a substance comprising the first material and the second material.

According to further second embodiments of the membrane, the spin-coated substance may comprise between 5% w/v and 15% w/v, preferably between 7 and 12% w/v, most preferably between 9 and 10% w/v of the first material and between 2 and 10% w/v, preferably between 5 and 8% w/v, most preferably between 6 and 7% w/v of the second material. In an exemplary first embodiment of the membrane, the membrane may comprise 9.35% of the first material and 6.34% of the second material.

According to further first and second embodiments of the membrane, the porous scaffold may have a thickness of less than 10 μm, preferably less than 5 μm, most preferably less than 1 μm. Furthermore, referring again to both the first and the second embodiments of the membrane, the porous scaffold may have a volume porosity of 1 to 50%, more preferably of 5 to 45%, even more preferably of 10 to 40%. Furthermore, the porous scaffold may have an area- or volume-weighted median pore diameter between 0.5 and 20 μm, preferably between 1 and 15 μm, even more preferably between 2 and 10 μm. Furthermore, the porous scaffold may have an elasticity, e.g. defined as the elastic modulus (or Young's modulus), of less than 5 MPa, more preferably less than 1 MPa, even more preferably less than 0.1 MPa, the latter being roughly two orders of magnitude larger than the elasticity of alveolar lung tissue, but at least 3- to 10-fold better than currently available membranes for ALI cell cultures.

According to further first embodiments of the membrane, the thickness of the filling layer may be equal to or smaller than that of the fibrous scaffold, preferably less than 70% of the thickness of the porous scaffold, even more preferably less than 30% of the thickness of the porous scaffold. The membrane according to first and second embodiments may be configured such that the duration for complete dissolution of the covering layer in water and/or a cell culture medium may be more than 12 hours and less than 2 weeks, preferably more than 1 day and less than 1 week, even more preferably more than 1.5 days and less than 4 days. However, the given time scales are of exemplary nature and merely provide a rough orientation for the dissolution times. In general terms, the membrane according to first and second embodiments is configured such that the time of dissolution of the filling layer from the membrane is of the order of time needed by the cells to grow into a confluent layer. The latter depends on cell type, in particular the type of cells and their specific proliferation rate on the membrane, number and density of cells initially seeded on the membrane and the volume fraction of the second material provided in or on the membrane. Furthermore, the dissolution time scale is further affected by physical inhibition of dissolution due to the thickness and porosity of the membrane, as not all of the gelatin is in contact with the liquid culture medium at the same time.

According to further first and second embodiments of the membrane, the first material may comprise a hydrophobic material, preferably polycaprolactone. Furthermore, the second material may be a wettable material and may preferably comprise gelatin. The wettability of the second material may translate into a water contact angle of less than 80°, preferably less than 70°, most preferably less than 50°, when a plane surface made of the second material is considered.

According to further first and second embodiments of the membrane, during the first phase it may comprise at least 10%, preferably at least 20%, even more preferably at least 30% of the second material by weight. During the second phase, the membrane according to further first and second embodiments may comprise a negligible fraction of the second material, i.e. 0% or at least close to 0% of the second material by weight, as it has been dissolved from the membrane.

According to various embodiments, a method of fabricating a biocompatible composite membrane is provided, in particular a membrane according to the second embodiment, the method comprising spin coating of a substance comprising the first material and the second material, wherein the second material forms conglomerates which are distributed in a layer comprising the first material. Due to the nature of this fabrication process, the conglomerates may be distributed in the layer comprising the first material in a chaotic or inhomogeneous manner.

For the manufacture of the second embodiment of the membrane, spin coating may be used to manufacture a thin, non-porous membrane. The membrane may have a thickness of 10 μm or less, more preferably of 5 μm or less, even more preferably of 1 μm or less. When seeded with cells, the membrane may provide for a defined growth of a confluent cell layer on its surface so that the cells grow directly at the ALI during the first phase. In particular, the membrane may be manufactured without use of any kind of cross-linkers which has the effect that the first material and the second material are not homogeneously mix during spin coating in the sense that they do not form a more or less homogenous blend of the two materials. Due to the separation of the material phases comprising the first material and the second material the membrane contains micrometer-sized “islands” of the second material embedded in a “sea” of the first material. During cell growth, which takes place during the first phase, the cell culture medium gradually dissolves the islands of the second material. This process turns the originally non-porous and rather stiff membrane which was well suitable for the first phase into a porous/permeable and more elastic membrane, which is well suitable for the second phase.

According to further embodiments of the method, the spin coated substance used in the method may correspond to the spin-coated substance of the second embodiments of the membrane as defined above.

The general rationale for the provision of the membrane according to various embodiments is the following. While the second material, e.g. gelatin, is often not elastic, it provides suitable conditions for initial cell growth. Additionally, the fact that it is soluble in cell culture medium results in gradual appearance of porosity when it is dissolved from the membrane according to various embodiments. The first material, e.g. PCL, on the contrary, is non-soluble in cell culture medium and provides elasticity and structural integrity of the membrane during cell-stretch conditions, but it is poorly wettable and hence not conducive to cell growth. Thus, it has been realized that mixing the first material and the second material with so to speak diametrically opposite properties when considered from a biological viewpoint could be a promising membrane concept satisfying the requirements of the first phase and the second phase of an experiment, as defined previously.

In this context it needs to be mentioned that spin coating has not been widely used for membrane fabrication mainly since it does not allow for fabrication of porous membranes. However, it is very useful for generating extremely thin membranes, with thicknesses of 10 μm and less, which can reduce membrane-induced biases.

Electrospinning is known to be used for manufacturing porous membranes. However, the achievable pore sizes according to prior art are too large for initial self-sealing without making the membrane much thicker than 20 μm. Using a thin sacrificial layer comprising the second water-soluble material with a thickness of approximately 0.5 μm or less on top of the porous scaffold provides a suitable substrate for initial cell adhesion and leads to the cells growing as a monolayer on the first (air-facing) surface of the membrane rather than a multilayer or a monolayer at least partially within the membrane. By adjusting the parameters of the water-soluble second material for the sacrificial layer, in particular its thickness and type, it is set to dissolve slowly enough to enable both initial cell growth and the formation of a confluent layer during the first phase, and, ultimately, dissolution of the second material introduces porosity leading to elasticity and permeability of the remaining membrane structure which are essential requirements for the second phase of the experiment in which cell stretch is observed under ALI conditions. In general, two types of gelatin as the second material may be used for the purpose of the present invention, type A and type B, which have different rheological properties. Type A, from porcine skin is obtained by acidic pretreatment. Type B is obtained by alkaline pretreatment. Even though both types may be used, type A may be more preferable for the construction of the membrane scaffold due to its larger number of carboxylic groups.

In summary, the following aspects are most relevant for providing optimal membrane characteristics for the two phases of cell culture experiments, as ensured by means of the membrane according to various embodiments of the invention. In the first phase, cells initially adhere to and grow on a non-porous, smooth membrane under submerged culture conditions and cells more easily adhere on the more wettable parts of the membrane comprising at least the second material. Once the cells proliferate, they secrete their own extracellular matrix (ECM) proteins, which allows the cells to also cover poorly wettable portions of the membrane according to various embodiments, such as the portions thereof comprising the first material. Ultimately, this leads to formation of a confluent cell layer covering the entire first (air-facing) side of the membrane. The lack of pores during the first phase prevents the cells from migrating into and through the membrane according to various embodiments to the other side thereof. In the course of the first phase, the second material is gradually dissolved in the cell culture medium. This process typically takes place over days, leaving behind pores, which has two important implications for the membrane. In the second phase, those pores provide the necessary porosity for air-lifting of the cell culture (ALI conditions) requiring permeability, i.e. contact of the cells on the air-facing side of the membrane with the culture medium on the second or basal side thereof to maintain cell viability. Moreover, the remaining porous membrane structure comprising the first material is more elastic than the solid membrane from the first phase and hence stretchable, yet provides enough structural integrity and biocompatibility for long-term stretch, e.g. cyclic stretch, without membrane fatigue during the second phase where cell stretch experiments take place under ALI conditions.

Through co-optimization of the parameters relevant to both phases of the experiments, as described above (e.g. elastic modulus, surface wettability, porosity and permeability of the membrane), using a design of experiment (DoE) approach, an optimal mixing ratio of PCL as the first material and gelatin as the second material has been established, namely 9.35% w/v of PCL and 6.34% w/v of gelatin, dissolved in TFE (2,2,2-trifluoroethanol) for the manufacture of a membrane according to the second embodiments of the present invention. Using this material mixture for the manufacture of the membranes according to various embodiments provides the advantages described above.

Overall, the membranes according to various embodiments, as described herein, satisfy all of the requirements regarding biocompatibility and cell growth, elasticity, maintenance of structural integrity under long-term cyclic stretch (at least up to 48 h) and permeability/porosity (for nutrient transport under ALI conditions). A further advantage provided by the inventive membrane is that there is no need for any kind of pre-processing/coating of the membrane with cell-proliferative agents (e.g. collagen-/fibronectin coating) to enable sufficient cell growth. The membrane, as manufactured, by its inherent biphasic nature by design, is capable of fully supporting physiological cell adhesion and cell proliferation. Furthermore, inadvertent adsorption of (mildly) hydrophobic drugs and/or proteins and cytokines may be avoided. At the same time, there is no leaching or migration of membrane molecules into the cell culture medium—in contrast to membranes based on PDMS that is known for significant amount of leaching of silicone in the surrounding culture medium. Last but not least, the membrane according to various embodiments is transparent and hence suitable for light microscopy (of the cells), which is an important aspect for current cell culture technologies.

The membrane according to various embodiments allows for a transition from an ultra-thin, wettable, non-porous and rather stiff membrane, as present in and optimally required for the first phase, into an ultra-thin, biocompatible, porous and elastic membrane, as present in and optimally required for the second phase. This transition is further advantageous in that the handling of the membrane, first and foremost the placing of the membrane in the membrane holder, is done while the membrane is still in its configuration for the first phase, i.e. while it is still rather stiff. This allows for reduced stiffness (enhanced elasticity) of the membrane in the second phase to extremely low levels without making membrane handling difficult.

The biphasic nature of the various embodiments of the membrane is designed to provide optimum conditions for the two distinctly different phases of typical cell culture experiments. As already mentioned, during the first phase attachment of the cells to the membrane and subsequent initial growth of the cells is ensured and the second phase relates to performing efficacy/toxicity experiments with a confluent cell culture model under cell-stretch and ALI conditions mimicking the alveolar air-blood barrier. The membrane according to various embodiments is particularly useful in combination with a bioreactor system which will be described below in more detail for real-time monitoring of the elasticity of the cells/membrane during the cell-stretching. None of the other ALI stretch cell systems described in the literature is reported to have the capability of membrane stretch/elasticity monitoring in real-time.

In various embodiments a bioreactor for investigating cells attached to an elastic membrane, preferably a biocompatible composite membrane according to various embodiments, as described above, under cyclic stretch conditions is provided. The stretch conditions preferably include cyclic stretch conditions. The bioreactor may be additionally used for culturing of the cells, in particular under ALI conditions. The bioreactor includes a housing, having a first portion and a second portion configured to be engaged with each other and defining an inner volume. The bioreactor further comprises the membrane, being positioned such that it divides the inner volume of the housing into a first volume, the first volume being in contact with a first surface of the membrane, and a second volume, the second volume being in contact with a second surface of the membrane. The bioreactor further comprises a first pressure sensor for determining a pressure in the first volume and a second pressure sensor for determining a pressure in the second volume. Furthermore, the bioreactor comprises means for actively, preferably cyclically, adjusting a pressure in the first volume or the second volume. The terms first and second volume may be understood as a first and second compartment of the bioreactor which are separated from one another by means of the membrane.

Either the first volume or the second volume may be fully or partially filled with a (cell) culture medium.

According to various embodiments of the bioreactor, the means for actively adjusting the pressure in the first volume or the second volume may comprise a fluid pump coupled to the respective volume via an access port. In one example, the fluid pump may be an air pump or an air flow regulation device and it may be used to adjust (increase or decrease) the air pressure in the corresponding volume by introducing air into it or withdrawing air from it. In another example the fluid pump may be a pump for liquids and it may be used to adjust the amount of liquid, e.g. culture medium, present in the corresponding volume by introducing extra liquid into it or withdrawing some liquid from it.

According to further embodiments of the bioreactor, the access port may be arranged in a portion of the housing defining the first volume and/or the second volume, the bioreactor further comprising a fluid reservoir coupled to the at least one access port of the bioreactor. The first pressure sensor and the second pressure sensor may be arranged either in the corresponding volume, i.e. the first or second volume, or the respective fluid reservoir coupled to that volume. The first and the second pressure sensor may be used to measure the pressure in the corresponding volume or in the fluid reservoir coupled thereto. In the case of a fluid reservoir coupled to the first volume, the fluid reservoir may only comprise a gas, such as air. In the case of a fluid reservoir coupled to the second volume, the fluid reservoir may comprise both a gas and a liquid. Accordingly, the bioreactor may comprise none, one or two fluid reservoirs. Under the assumption of incompressibility of the liquid and compressibility of the gas (here air), a variation of the pressure in the first volume or the second volume corresponds to a variation of the pressure in the corresponding fluid reservoir (if present), irrespective of the location of the source generating the variation of the pressure, i.e. first or second volume or the corresponding fluid reservoir. The fluid reservoir coupled to the second volume is preferentially provided with a head space which is not filled with liquid (here: culture medium) but with gas (here air), such that the second pressure sensor may be used to measure the pressure in the head space of the fluid reservoir. According to further embodiments of the bioreactor, the means for adjusting a pressure in the first volume or the second volume may comprise at least one portion of the housing of the bioreactor which is movable with respect to the remaining housing such that the respective volume (i.e. first or second volume) of the bioreactor may be adjusted. From the point of view of the cell stretch experiments which may be performed by means of the bioreactor, a change of the actual spatial size of the volume may be used to adjust the pressure in the first and/or second volume.

The strain/elasticity monitoring experiments performed with the bioreactor are based on application or change of pressure in one of the two volumes and observation of the resulting pressure change in the other volume. The change of the pressure in the other volume may be either determined by measuring the pressure directly in the other volume or by measuring the pressure in a fluid reservoir coupled to the other volume. As already mentioned, the application of pressure in the one volume may also include changing the actual spatial size of the volume such that the air or the liquid comprised therein is compressed or displaced.

In various embodiments a method for investigating cells attached to a membrane is provided, wherein the method may be preferably performed by means of the bioreactor described herein. The method comprises determining the elastic modulus or stiffness of the membrane while actively adjusting the pressure in the first volume or in the second volume. Determining the elastic modulus of the membrane may include determining the elastic deformation or the change of the profile of the membrane which is subject to stretch. Determining the elastic modulus of the membrane may include directly or indirectly measuring the volume of the displaced liquid medium which causes a three-dimensional stretch of the membrane. In particular, determining the elastic modulus of the membrane may include time-resolved, preferably real-time pressure sensing in the first and second volume. From the obtained pressure values, which under cyclic stretch conditions can be described by an amplitude and frequency, the elastic modulus of the membrane may be calculated. In order to determine the amplitude and frequency of distension or stretch of the membrane, either the first pressure or the second pressure needs to be monitored/measured. Determination of the elastic modulus of the membrane is based on monitoring the first pressure and the second pressure. Furthermore, the method may include, as a preparatory step, arranging the membrane in a membrane holder of the bioreactor.

According to further embodiments of the investigating method, investigating the cells may include measuring the pressure in the respectively other volume, preferably over time while, the pressure in the first or second volume is adjusted, preferably cyclically and preferably with positive differential pressure. The reference point for the determination of the differential pressure may be the ambient pressure. Thus, the adjustment of the pressure with a positive differential pressure may include increasing the pressure in the respective volume above ambient pressure and then returning back to ambient pressure.

According to further embodiments, the investigating method may further comprise providing a liquid in the second volume such that the second volume is at least partly filled with the liquid and the liquid is in direct contact with the membrane. The partial filling of the second volume with the liquid leaves at least a portion of the second volume empty, thus allowing for the formation of an air-filled headspace. The air-filled headspace may be located directly in the second volume, for example, in the fluid reservoir which is coupled to the second volume. The air-filled headspace, which is compressed and decompressed in response to a pressure change in the bioreactor, e.g. in the first volume thereof, may be used to determine the pressure in the second volume. The liquid may be a cell culture medium.

According to further embodiments of the investigating method, the first and the second volume may be at least partly filled with gas, preferably air.

According to further embodiments of the investigating method, a liquid reservoir may be coupled to the housing, e.g. via the access port provided on the bioreactor, and may be in fluid communication with the second volume, the liquid reservoir being partly filled with the fluid. According to yet further embodiments of the investigating method, a (separate) liquid reservoir filled with air only may be coupled to the first volume via a further access port.

According to further embodiments of the investigating method, the second pressure sensor may be provided in a headspace of the liquid reservoir which is filled with a gas, preferably air.

According to further embodiments of the investigating method, the second volume may be partly filled with the liquid such that in an equilibrium state of the membrane the second volume comprises a headspace which is filled with a gas, preferably air. The equilibrium state of the membrane may refer to a state in which the pressure within the reactor corresponds to the ambient pressure, i.e. when no additional pressure is applied to the membrane.

According to further embodiments of the investigating method, the second pressure sensor may be provided in the second volume. Here, the air-filled region which may be present in the second volume, formed between the bottom surface of the membrane and the surface of the liquid provided in the second volume, may be used to determine the pressure within the second volume.

According to further embodiments of the investigating method, the pressure in the second volume may be adjusted by applying pressure to the fluid provided in the second volume. In order to adjust the pressure in the second volume, at least one portion of the first part of the housing of the bioreactor may be moved with respect to the remaining housing such that the size of the volume of the bioreactor is changed, preferably reduced. Alternatively, the pressure may be applied by introducing more fluid into the second volume through the access port from the fluid reservoir.

According to further embodiments of the investigating method, the cells to be investigated may be arranged on the first surface of the membrane. Such an experimental setup may provide the basis for lung experiments where the cells are examined under ALI conditions.

According to further embodiments the investigating method may comprise determining the pressure in one of the first and second volume while adjusting the pressure in the respectively other volume, preferably using positive differential pressure. In a preferred experimental setup, the investigating method may comprise determining the pressure in the second volume while adjusting the pressure in the first volume.

According to further embodiments of the investigating method, the gas-filled portions of the first volume and of the second volume may be larger than the maximum volume displacement of the membrane during adjusting the pressure in the first or the second volume.

Generally, the pressures measured in the first volume and in the second volume can be used to simultaneously determine amplitude and frequency of strain applied to the cells and the elastic modulus (Young's modulus) of the membrane to which the investigated cells are attached, preferably of the cells themselves.

Stretch frequency and stretch amplitude as well as membrane elasticity have been shown to affect cellular response parameters. The method according to various embodiments for investigating cells attached to the membrane allows for continuous monitoring of those parameters during cell-stretch experiments, wherein those parameters may be used for quality control as well. Therefore, the various embodiments of the method for investigating cells attached to the membrane may be seen to correspond to a method for monitoring both the strain profile (amplitude, frequency) and mechanical properties (elastic modulus) of the (cell covered) membrane during cyclic stretch conditions. The inventive method takes advantage of the well-defined relationship between the pressures in the two volumes defined within the housing of the bioreactor, which are separated by the stretchable membrane. For a slightly perforated membrane as used herein, which allows diffusive exchange of biomolecules between cells located on one side (usually top side) thereof and the culture medium located on the other side (usually bottom side) thereof, the membrane is brought in contact with the culture medium which corresponds to an incompressible liquid in order to “seal” the pores of the membrane. The liquid is more viscous than air and the pores are too small and/or the cell-covered pores are too well sealed to enable convective transport of the liquid itself. In consequence, the liquid medium is prevented from being transported across the (cell-covered) membrane during the stretch cycles.

In the following, embodiments of the present invention will be described in more detail with reference to the appended figures.

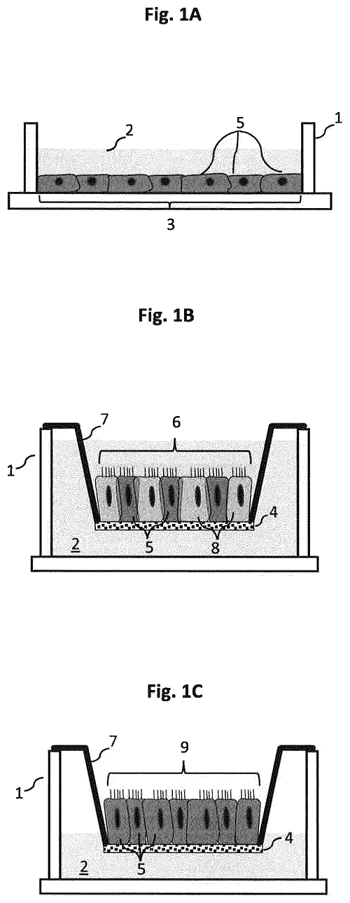

FIG. 1A shows a cell culture under submerged cell culture conditions.

FIG. 1B shows a cell culture under ALI conditions during the first phase of cell experiments with the membrane according to various embodiments in its first configuration.

FIG. 1C shows a cell culture under ALI conditions during the second phase of cell experiments with the membrane according to various embodiments in its second configuration.

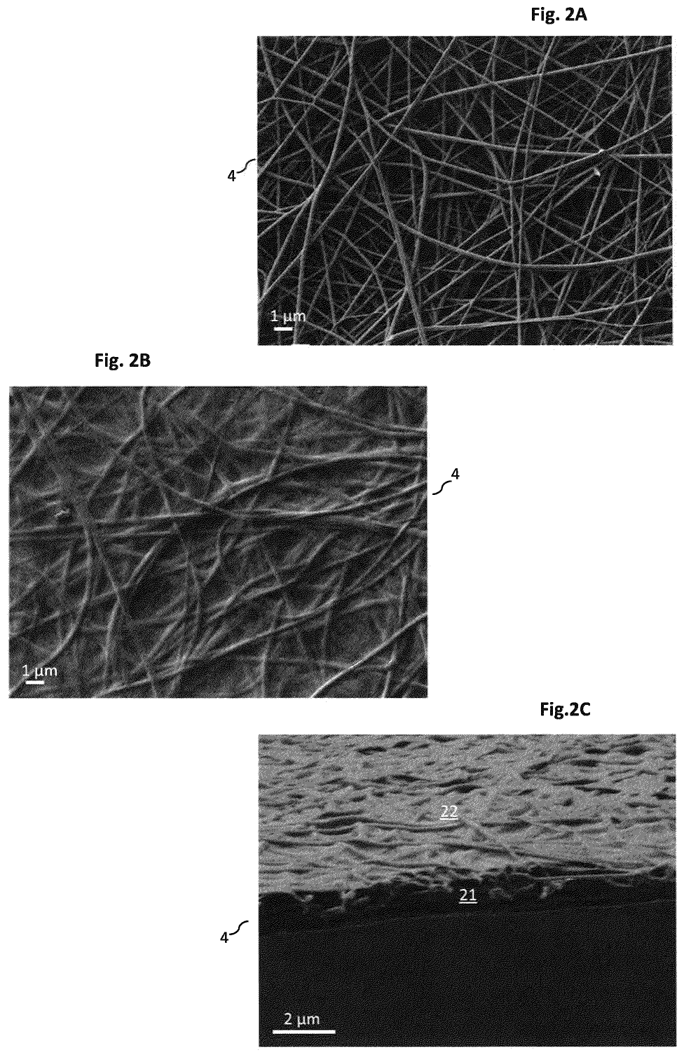

FIG. 2A is a Scanning Electron Microscopy (SEM) image showing the structure of an exemplary membrane according to various first embodiments during the second phase of cell experiments (second configuration).

FIG. 2B is an SEM image showing the membrane structure of the membrane according to various first embodiments from FIG. 2A during the first phase of cell experiments (first configuration).

FIG. 2C is a cross-sectional SEM image of an exemplary membrane according to various first embodiments during the first phase of cell experiments.

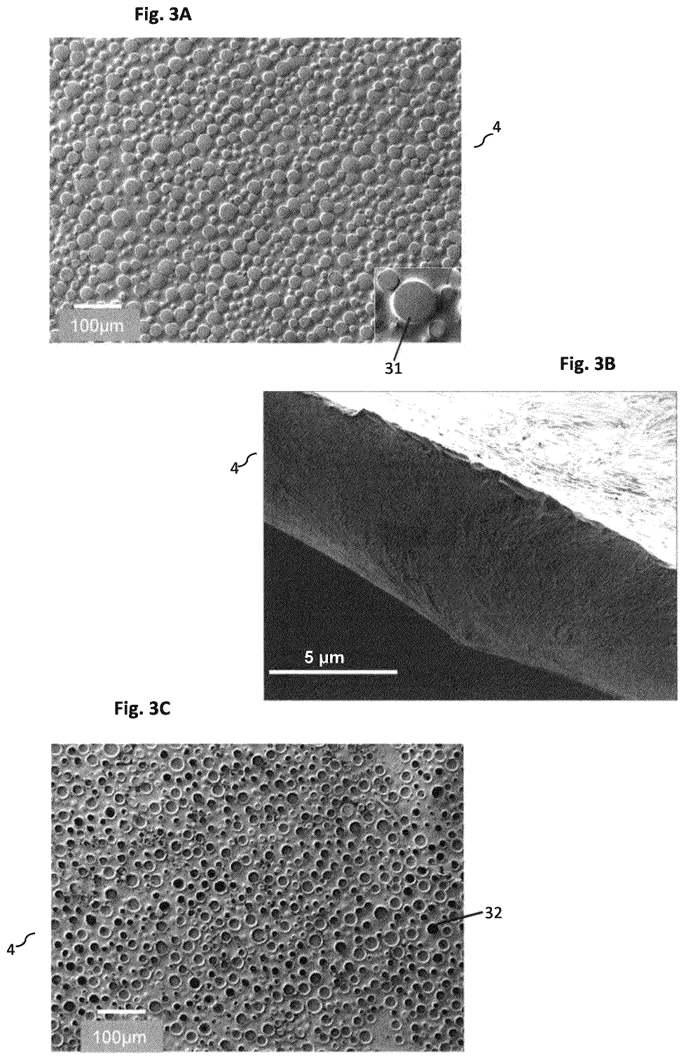

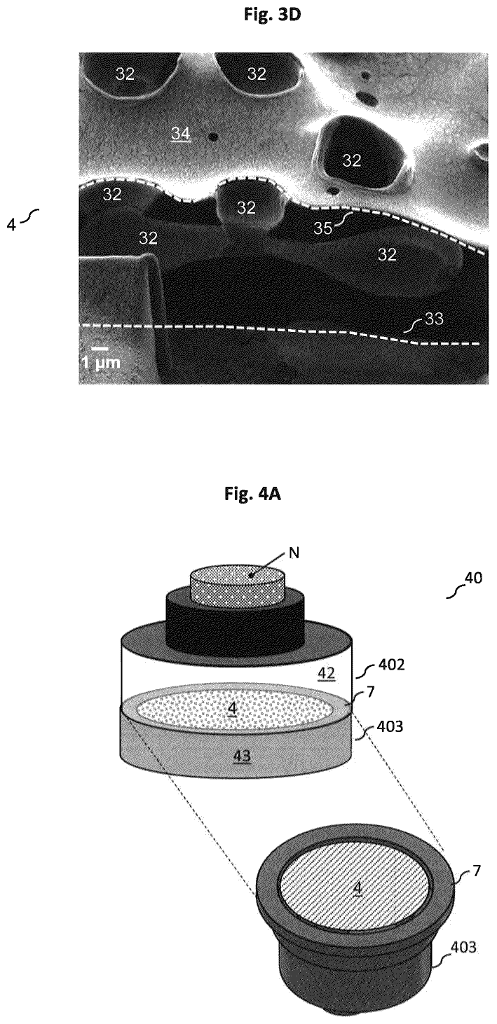

FIG. 3A is a SEM image showing the structure of an exemplary membrane according to various second embodiments in its configuration for the first phase of cell experiments.

FIG. 3B is a cross-sectional SEM image of an exemplary membrane according to various second embodiments in its configuration for the first phase of cell experiments as depicted in FIG. 3A.

FIG. 3C is an SEM image showing the membrane structure of the membrane according to various second embodiments as depicted in FIG. 3A in its configuration for the second phase of cell experiments.

FIG. 3D is a cross-sectional image taken by focused ion beam (FIB) SEM tomography of an exemplary membrane according to various second embodiments in its configuration for the second phase of cell experiments as depicted in FIG. 3C.

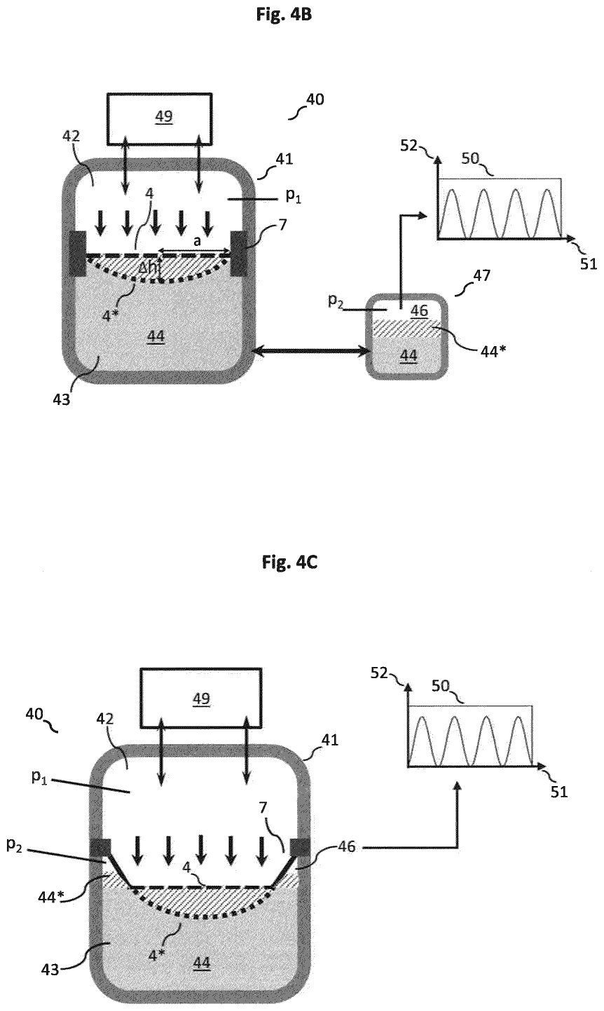

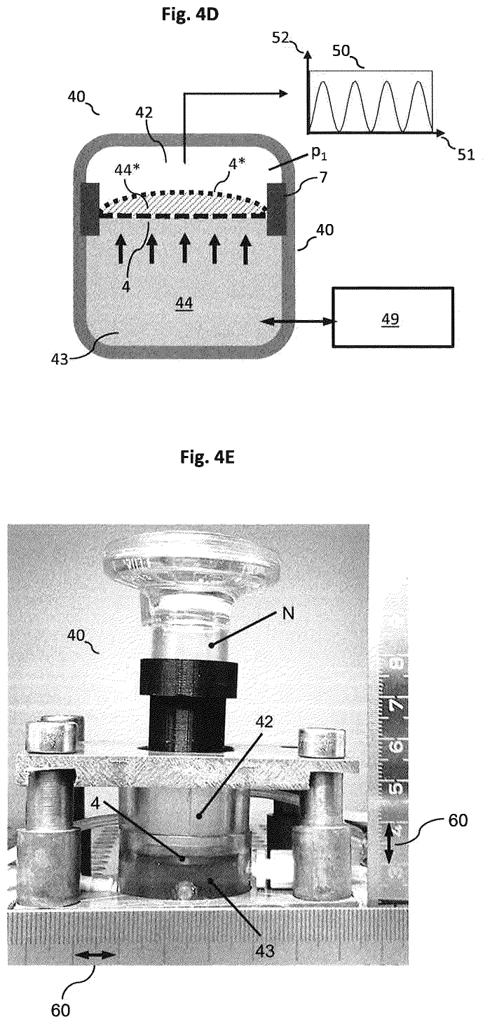

FIG. 4A shows an embodiment of a bioreactor according to various embodiments.

FIG. 4B shows an experimental setup based on an exemplary bioreactor according to various embodiments.

FIG. 4C shows a further experimental setup based on an exemplary bioreactor according to various embodiments.

FIG. 4D shows a further experimental setup based on an exemplary bioreactor according to various embodiments.

FIG. 4E shows a photo of an embodiment of a bioreactor according to various embodiments.

FIG. 1A shows a standard cell culture 3 under submerged cell culture conditions, as known from the state of the art. In experimental setups of that nature, the cell culture 3 comprising a layer of cells 5 is arranged at the bottom of a suitable container 1, e.g. a plastic well, and is completely covered with the cell culture medium 2. As already noted in the introductory section, from the perspective of the cells 5, the submerged cell culture 3 does not replicate the physiological conditions of the epithelium in the lungs and is therefore for predictive inhalation therapy or inhalation toxicology experiments than more physiologic cell culture models. In contrast to the conditions in a real lung, aerosolized drugs or toxins cannot be deposited directly onto the lung epithelium but have to diffuse through and potentially interact with the cell culture medium 2 first in order to reach the submerged cells 5.

To remedy the deficiency inherent in the submerged cell culture 3 depicted in FIG. 1A, the air liquid interface (ALI) has been developed. The corresponding experimental setup is shown in FIG. 1C. In order to simulate an ALI for the cells, the cells 5 are also located in a suitable container 1. However, in contrast to the submerged cell culture 3, the cells 5 in an ALI culture 9 are not submerged in the cell culture medium 2, but instead they are cultured on the top side of a suitable membrane 4, preferably the membrane according to various embodiments, and the cells on the membrane being in contact with the cell culture medium 2 provided on the opposite side of the membrane 4. The membrane 4 is held in place by a membrane holder 7. By arranging the cells 5 on the membrane 4, a more physiological cell culture can be obtained: The top, air-facing surface of the cells 5 corresponds to the apical surface of the epithelial cell culture which in real conditions is exposed to the external environment (air-filled cavity) of an internal organ inside the body, e.g. the alveolus inside the lung, whereas the bottom surface of the ALI cell culture 9 corresponds the basal side which is located closer to the surface of the membrane 4 and also closer to the cell culture medium 2. The ALI setup shown in FIG. 1C is more biomimetic than the submerged cell culture setup depicted in FIG. 1A since it provides more physiological drug/toxin delivery and cell conditions by allowing for direct aerosol deposition from the air onto the confluent cell layer 9 and polarization of the confluent cell layer 6 (i.e. air-facing side of cells is biologically different—protected against drying out at the air—from liquid/medium facing side), respectively.

The more physiologic ALI cell culture as required for the second phase of the ALI experiment (FIG. 1C) requires an initial cell seeding and growth phase, which was referred to as first phase of the ALI experiment above and is shown in FIG. 1B. During this phase a relatively small number of cells 5 is seeded on the membrane 4 and cultured under submerged culture conditions with medium on the apical (top) and basal (bottom) side. Under these conditions, the cells 5 of the initially non-confluent cell layer 6, i.e. a cell layer having gaps 8, will proliferate and the resulting new cells will fill the gaps 8 until the confluent cell layer 9 is formed, as shown in FIG. 1C. During cell proliferation cells are motile and they may migrate into and/or through the membrane 4, if the membrane 4 contains large enough pores (typically larger than ca. 3 μm), which is not desirable since in the lung the epithelial layer is formed on the apical side only. Hence, during the first phase of the ALI cell experiment a non-porous membrane 4, such as the membrane according to various embodiments in its first configuration, is beneficial. Once a confluent cell layer 9 has been formed, all cells 5 have contact with neighboring cells. This inhibits motility of the cells and thus the membrane 4 can now have pores without cells migrating and/or though the membrane 4 according to various embodiments in its second configuration, which is the prerequisite for air-lifting of the cells as required for the second phase of the ALI cell experiment depicted in FIG. 1C.

As already described, the membrane according to various embodiments has been conceived in order to provide an optimal environment to the cells 5 during the two distinct phases of cell stretch experiments. FIGS. 2A-2C are SEM images showing the structure of an exemplary membrane 4 according to various first embodiments which may be preferably manufactured by electrospinning, as described above. FIG. 2A shows the structure of an exemplary membrane 4 according to various first embodiments configured for the second phase of cell experiments (FIG. 1C), while FIG. 2B shows the same membrane 4 configured for the first phase of cell experiments (FIG. 1B). The membrane 4 according to various first embodiments comprises electrospun fibers comprising at least PCL as the first material, preferably a mixture of PCL and gelatin as the second material. The average fiber diameter is 182±7 nm. In the image of FIG. 2B the membrane 4 is shown in a state after it has been spin-coated with a covering layer of the second material which plays the role of the sacrificial material. That is, the layer of the second material is dissolved, in analogy to the membrane according to various second embodiments, when the membrane 4 according to various first embodiments transitions from its first configuration to its second configuration. The scale bar in both figures is 1 μm.

FIG. 2C is an SEM image showing a cross-sectional view of an exemplary membrane 4 according to various first embodiments in its first configuration for use in the first phase. The scale bar in the image represents a scale of 2 μm. The membrane 4 comprises the scaffold 21 or the electrospun membrane comprising electrospun fibers which themselves comprise a mixture of PCL as the first material and gelatin as the second material. The scaffold 21 is covered with a covering layer 22 (sacrificial layer) comprising gelatin as the second material. The exemplary membrane 4 shown in FIG. 2C in its configuration for the first phase has an average thickness of 1.30 μm±0.16 μm. The same membrane 4 in its configuration for the second phase, i.e. without the covering layer 22, as shown in FIG. 2A, has an average thickness of 0.98±0.16 μm.

FIG. 3A is an SEM image showing the structure of an exemplary membrane 4 according to various second embodiments during the first phase of cell experiments (FIG. 1B) which may be preferably manufactured by spin coating as described above. The membrane 4 according to various second embodiments comprises an inhomogeneous mixture of the first material and the second material, wherein the second material is provided in the form of conglomerates of various sizes. In the bottom right corner of the SEM image an enlarged portion of the membrane 4 is shown. In the enlarged image, an agglomeration of conglomerates 31 of the second material can be seen, which may be seen to correspond to circular islands, which extend into the membrane 4 to form a three-dimensional interconnected network of conglomerates as illustrated below (FIG. 3C and 3D).

FIG. 3B is an SEM image showing a cross sectional view of an exemplary membrane 4 according to various second embodiments in its configuration for use in the first phase (FIG. 3A). As can be deduced from the scale bar having a length of 5 μm, the thickness of the membrane 4 is approximately 6 μm. The lack of visible conglomerates 31 in the image as seen in FIG. 3A originates from the difference in resolution of both figures (indicated by scale bars) and the change in perspective requiring tilting the membrane 4 as compared to its orientation relative to the imaging sensor in FIG. 3B. This results in loss of some information, such as the visualization of the conglomerate structure of the membrane 4, which is evident in FIG. 3A.

FIG. 3C is an SEM image showing the membrane from FIG. 3A, but in its configuration for use in the second phase (FIG. 1C). The structure of the membrane 4 configured for the second phase is characterized by pores 32, which resemble crater-like structures. The pores correspond to void spaces in the membrane 4 which are formed after removal of the conglomerates 31 of the second material from the membrane 4. The removal of the conglomerates 31 of the second material, i.e. the transition of the membrane 4 from its configuration for the first phase (FIG. 1B) to its configuration for the second phase, takes place trough dissolution of the conglomerates 31 of the second material. For that reason, the islands or conglomerates 31 of the second material may be seen to correspond to a sacrificial material which is removed by dissolution from the membrane 4 to introduce porosity to the membrane 4. In doing so, not only porosity is introduced but also the elastomechanical properties of the membrane 4 are changed i.e. the elasticity of the membrane is increased. The scale bars shown in FIG. 3A and 3C are 100 μm long. As mentioned above, the membrane 4 according to various second embodiments may be preferably manufactured by spin coating and it may comprise a basal layer comprising PCL as first material and conglomerates of gelatin as the second (sacrificial) material.

FIG. 3D is a cross-sectional image taken by focused ion beam (FIB) SEM tomography of an exemplary membrane according to various first embodiments in its configuration for the second phase of cell experiments (FIG. 3C). As can be deduced from the scale bar having a length of 1 μm, the large pores 32 form a three-dimensional network of pores 32 with a diameter of approximately 1-5 μm throughout the entire membrane 4. In the image, the pores 32 are shown to be open to the first (apical) surface 34 of the membrane 4. The second (basal) surface 33 of the membrane 4 is indicated by the lower dashed line. The region between the upper dashed 35 line and the lower dashed line 33 is the cross-view onto the membrane 4.

In FIG. 4A, a schematic depiction of a bioreactor 40 according to various embodiments is shown. The bioreactor 40 may be subdivided structurally into a first volume 42, which is in contact with the first surface of the membrane 4 and practically defined by the first portion 402 (e.g. a top portion) of the housing of the bioreactor 40, and a second volume 43, which is in contact with the second surface of the membrane 4 and practically defined by a second portion 403 of the housing of the bioreactor 40. The membrane 4 is held by a membrane holder 7 such that it is arranged between the first volume 42 and the second volume 43. A nebulizer N is arranged above the first volume. The nebulizer N is configured to generate a mist which may be deposited on the cells arranged on the first surface of the membrane 4 for drug/toxin experiments. The nebulizer N may be also configured to provide a continuous measurement of the pressure in the first volume 42. During the experiments, the culture medium is provided to the second volume 43, whereas the first volume 42 is filled with air. In the lower portion of FIG. 4A an additional perspective view on the membrane holder 7 and the second portion 403 of the housing is depicted. The bioreactor 40 may have a generally cylindrical cross-section.

FIGS. 4B-4D show different configurations of the bioreactor 40 according to various embodiments for investigating the membrane and consequently the cells arranged thereon. In general, as already described, the bioreactor 40 comprises a housing 41, having the first portion and the second portion configured to be engaged with each other and defining an inner volume. Inside the housing 41, the bioreactor 40 comprises the membrane 4 according to various embodiments. In a top view, the bioreactor 40 may have a cylindrical geometry. The membrane 4 is held or spanned across the inner volume of the bioreactor 40 such that it divides the inner volume of the housing into the first volume 42, the first volume 42 being in contact with a first surface of the membrane 4, and a second volume 43, the second volume 43 being in contact with the second surface of the membrane 4. A membrane holder 7 may be used to install the membrane 4 inside the housing 41, wherein the membrane holder 7 may comprise a plastic ring in which the membrane 4 is installed. The membrane holder 7 may be provided inside the housing 41 or may be an adapter which is arranged, together with the membrane 4 installed therein, inside the bioreactor 40. As can be taken from FIGS. 4B-4D, the first surface of the membrane 4 corresponds to its upper surface and may be seen to correspond to the apical side thereof, wherein its second surface corresponds to its bottom surface and may be seen to correspond to its basal side. The cell culture arranged on the membrane 4 is not shown in FIGS. 4B-4D. The bioreactor 40 further comprises a first pressure sensor for determining a first pressure p₁ in the first volume 42 and a second pressure sensor for determining a second pressure p₂ in the second volume 43. The first pressure sensor may be installed in the first volume 42 of the bioreactor 40, the second sensor may be installed in the second volume 43 of the bioreactor 40 or in an air-filled headspace 46 of a separate reservoir which is coupled to the second volume 43. The bioreactor 40 further comprises a means 49 for actively, preferably cyclically, adjusting the pressure in the first volume 42 or the second volume 43 (pressure unit in the following). During the cell stretch experiments, a cell culture medium 44 is provided in the second volume 43, wherein the bottom surface of the membrane 4 is in contact with the cell culture medium 44. In general, the bioreactor 40 according to various embodiments may be used for investigating cells attached to or seeded on the membrane 4 under (preferably cyclic) stretch conditions at ALI. The membrane 4 used for this purpose is the biocompatible composite membrane according to various embodiments.

In the embodiment of the bioreactor 40 shown in FIG. 4B, a reservoir for a liquid 47 (liquid or medium reservoir) is provided and coupled to the second volume 43 of the bioreactor 40 via a connecting tubing. At the same time, the air-filled headspace in the liquid reservoir 47 is couped to the first volume 42 via the liquid-filled volume comprising the second volume 43, the liquid-filled part of the volume of the fluid reservoir 47 and the connecting tubing. The pressure unit 49 corresponds to an air flow regulation means, e.g. a compressor, and is used to increase the first pressure p₁ above ambient pressure in a cyclic manner. The application of a positive pressure to the top side of the membrane 4 causes deformation of the membrane 4 which in turn leads to a displacement of a certain volume of the culture medium 44. Due to its incompressibility, the culture medium 44 is transferred into the medium reservoir 47 where the second pressure p₂ is monitored by the second pressure sensor. In FIG. 4B (as well as in FIGS. 4C and 4D) the membrane 4 is also indicated in its stretched state and denoted by reference sign 4*. When no pressure is applied to the membrane 4, i.e. when the first pressure p₁ corresponds to the ambient pressure, the membrane 4 is in its unstretched or equilibrium state, resting on the surface of the culture medium 44. In the experimental setup shown in FIG. 4B, the culture medium 44 fills the entire second volume 44. That is, the entire bottom surface of the membrane 4 is in contact with the culture medium 44. The fluid reservoir 47 may be installed at an appropriate elevation relative to an access port at which the fluid reservoir 47 is coupled to the second volume 43 of the bioreactor 40 in order to adjust the level of the culture medium 44 in the second volume 43 such that no hydrostatic pressure is exerted on the membrane 4. In addition, the fluid reservoir comprises an air-filled headspace 46. The arrows in FIG. 4B indicate the direction in which the force exerted by the increased pressure p₁ generated by the pressure unit 49 acts on the membrane 4. In FIG. 4B the dashed volume 44* represents the portion by which the culture medium 44 inside the liquid reservoir 47 fluctuates, caused by the stretch of the membrane 4 which displaces the correspondingly equal volume of culture medium 44 from the second volume 43 and then, when it relaxes to its equilibrium state, it allows that same amount of culture medium 44 back into the second volume 43. The fluctuation of culture medium 44 inside the fluid reservoir 47 translates into a fluctuation of the second pressure p₂ as the air-filled headspace is being compressed and decompressed by the relocation of culture medium 44 from and back into the second volume 43. The inset 50 indicates the resulting cyclic change of the second pressure p₂ in the air-filled headspace 46, with the x-axis 51 denoting time and the y-axis 52 denoting pressure.

In the alternative embodiment of the bioreactor 40 shown in FIG. 4C the second pressure sensor is arranged in the second volume 43 of the bioreactor 40 such that the second pressure p₂ is measured directly in the second volume 43. In order to facilitate measurement of the second pressure p₂, the culture medium 44 does not fill the entire second volume 43 such that an air-filled headspace 46 is present in the second volume 43. The inset 50, which has been introduced in FIG. 4B, indicates that the pressure monitoring is performed based on the second pressure p₂. Also indicated is the dashed volume 44* by which the air-filled headspace 46 fluctuates due to the stretch of the membrane 4 into its stretched state 4* and the resulting change of the level of the culture medium 44 which compresses the air in the air-filled headspace 46. In FIG. 4C, the membrane holding means 7 includes a tapered membrane holder (similar to standard transwell inserts) which is made of an airtight material. Therefore, the air-filled headspace 46 is held airtight or sealed, so to speak, by the culture medium sealing the pores of the cell-covered membrane 44. The pressure unit 49 may be the same as in the configuration shown in FIG. 4B. The arrows in FIG. 4C indicate the direction in which the force exerted by the increased pressure p₁ generated by the pressure unit 49 acts on the membrane 4.

In the further embodiment of the bioreactor 40 shown in FIG. 4D, the first pressure p₁ is increased by applying pressure directly to the culture medium 44 by means of a suitably configured pressure unit 49. In this embodiment, the additional pressure is applied to the second volume 43 and the first pressure p₁ in the first volume 42 is monitored. For example, the pressure unit 49 may be an actuator which is configured to move a part of the housing 41 of the bioreactor 40, e.g. a portion of its bottom or side wall, thus applying pressure to the culture medium 44. Alternatively, the pressure unit 49 may include a fluid reservoir in connection with a pump which is configured to provide additional culture medium 44 into the second volume 43. By applying pressure to the culture medium 44, the membrane 4 is brought into its stretched state 4*. The arrows in FIG. 4D indicate the direction in which the force exerted by the pressure of the culture medium 44 acts on the membrane 4. As indicated by the inset 50, the pressure monitoring is performed based on the measurement of the first pressure p₁ in the first volume 42.

Monitoring of the pressure over time allows for monitoring of the pressure-based strain/elasticity relation of the membrane 4. In the embodiments of the bioreactor 40 shown in FIGS. 4B and 4C the pressure monitoring is performed by observing the second pressure p₂ and in the embodiment of the bioreactor 40 shown in FIG. 4D the pressure monitoring is performed by observing the first pressure p₁.

In general, stretch and the elastic modulus of the cell-covered or the plane membrane 4 can be measured using two pressure sensors (e.g. PMX5050, Freescale Semiconductor) which are provided in the apical compartment (first volume) and the basal compartment, respectively, i.e. in the air volume in the liquid reservoir 47, as shown in FIG. 4B, or in the air volume 46 provided directly in the second volume 43, as shown in FIG. 4C. In the embodiment shown in FIG. 4D, the second pressure p₂ may be determined from the direct application of pressure to the culture medium 44 or the displaced medium volume 44* is known from the operating conditions of the pressure unit 49. In general, independent of the actual setup chosen for the pressure measurement, from the difference of the elastic modulus of the cell-covered and the blank membrane 4, the elastic modulus of the cell layer itself can be calculated.

FIG. 4E shows an image of an exemplary bioreactor 40. The bioreactor 40 has a cylindrical shape and the middle and lower portion of the housing comprising the first volume 42 and the second volume 43 with a diameter of approximately 3 cm, respectively. The membrane 4 which is inserted in the housing may have a diameter of that order as well. The nebulizer N is arranged a few centimeters above the top of the first volume 42 to provide for a homogenous deposition of liquid aerosol onto the membrane 4 and the cells arranged thereon. The scale bars shown at the right side and at the bottom of the image are centimeter scales, wherein additional double-arrows 60 have been added, each marking a length of 1 cm.

In the following, the monitoring method will be explained based on the exemplary setup of the bioreactor 40 according to various embodiments shown in FIG. 4B. Once a positive pressure is applied to the first volume 42, the membrane 4 expands or is stretched into its stretched form 4* and pushes the culture medium 44 from the second volume 43 into the medium reservoir 47. The air-filled part of the second volume 43, which is represented by the air-filled headspace 46 in the embodiments shown in FIG. 4B and FIG. 4C, is compressed (ΔV) and leads to an increase of pressure Δp₂=p₂−p₀ in the headspace of the second volume, wherein p₀ corresponds to the pressure in both the first volume 42 and the air volume 46 in the second volume when the membrane is in a relaxed state. The volume change ΔV may be calculated from

$$Δ V = {V}_{2 , 0} \frac{Δ {p}_{2}}{{p}_{0}} , \qquad ( 1 )$$

wherein V_{2,0} corresponds to the air-filled part of the second volume 43 (i.e. its initial size) when no pressure is applied by the pressure unit 49, which is represented by the air-filled headspace 46 in the embodiments shown in FIGS. 4B and 4C. For the analysis, the shape of the stretched membrane 4* is approximated by a half dome geometry.

This volume change ΔV is caused by the membrane displacement, which may be calculated from the volume of a spherical cap, which may be calculated from the radius a of the base of the dome (here: a is the radius of the membrane 4 prior to application of a pressure by pressure unit 49) and the height Δh of the dome for the membrane in its stretched state 4* (both a and Δh are indicated in the embodiment of the bioreactor depicted in FIG. 4B) in the following manner:

$$Δ V = π Δ h ( \frac{{a}^{2}}{2} + \frac{Δ {h}^{2}}{6} ) ) . \qquad ( 2 )$$

Since ΔV is known from the first equation and a is known from the geometry of the membrane 4, the height of the dome Δh can be derived from this equation and thus the relative change in the membrane area transitioning from its non-stretched 4 into its stretched state 4* may be calculated (see equation 4).

The elastic modulus (Young's modulus) E, measured in kPa, of the membrane 4 can then be calculated from the following equation

$$E t = Δ {p}_{12} \frac{3 a ( {( \frac{Δ h}{a} )}^{2} + 1 )}{4 \frac{Δ h}{a} ( 1 - \frac{1}{{( 1 + {( \frac{Δ h}{a} )}^{2} )}^{3}} )} , \qquad ( 3 )$$

For membrane only

$${E}_{m} {t}_{m} = Δ {p}_{12 \_ m} \frac{3 a ( {( \frac{Δ {h}_{m}}{a} )}^{2} + 1 )}{4 \frac{Δ {h}_{m}}{a} ( 1 - \frac{1}{{( 1 + {( \frac{Δ {h}_{m}}{a} )}^{2} )}^{3}} )} \qquad ( 3 a )$$

For cell-covered membrane

$${E}_{m} {t}_{m} + {E}_{c} {t}_{c} = Δ {p}_{1 {2}_{m + c}} \frac{3 a ( {( \frac{Δ {h}_{m + c}}{a} )}^{2} + 1 )}{4 \frac{Δ {h}_{m + c}}{a} ( 1 - \frac{1}{{( 1 + {( \frac{Δ {h}_{m + c}}{a} )}^{2} )}^{3}} )} \qquad ( 3 b )$$

where the subscripts m and m+c refer to parameters during membrane only (no cell layer on membrane) and cell-covered membrane stretch experiments, respectively, while subscript c denotes parameters related to the cell layer only. Δp₁₂ is the difference between p₁ and p₂ (p₁ and p₂ are indicated in the embodiments of the bioreactor depicted in FIG. 4B and 4C) and corresponds to the force per area which is applied to the membrane 4, Δh is the height of the dome-shaped membrane, i.e. the elastic displacement (amplitude) of the center point of the membrane 4 during stretch, a is the radius of the non-stretched membrane 4 and its thickness tin its stretched state (at any given Δp₁₂). From the above equation, Young's modulus E of the membrane 4 can be calculated for each stretch cycle. The elastic modulus of the cell-layer itself (E_{c}) can be calculated from the difference of the elastic modulus of the cell-covered membrane (E_{c+m}) and that of the blank membrane (Eₘ) here expressed as difference of equation 3b and 3a.

Moreover, the change in (amplitude of) membrane surface area during stretch, often referred to as amplitude of membrane stretch, can be calculated according to

$$Δ S = {{S}_{0} ( \frac{Δ h}{a} )}^{2} = π Δ {h}^{2} , \qquad ( 4 )$$

where S₀ is the surface area of the membrane 4 in its relaxed state (S₀=πa²).

This mathematical approach may be used with the embodiments of the bioreactor 40 shown in FIG. 4B and FIG. 4C. In FIG. 4C, the medium reservoir 47 from FIG. 4B is eliminated and the second pressure p₂ is measured in the air-filled headspace 46 which is enclosed by culture medium 44, a portion of the inner wall of the second volume 43 of the housing 41 and the bottom side of the tapered membrane holder 7. Thus, the air-filled headspace 46 functions as a probe provided in the second volume 43 or in the fluid reservoir 47 coupled to the second volume 44 which is used to determine the second pressure P₂.

In FIG. 4D, the real-time stretch profile of the membrane 4 can be monitored in analogy to the other two embodiments by realizing that the profile of the stretched membrane 4* is inverted and will thus be observed as extending into the first volume 42 and not into the second volume 43, as is the case in FIG. 4B and FIG. 4C. Essentially, the embodiment of the bioreactor shown in FIG. 4D corresponds to an inverse of the bioreactor 40 shown in FIG. 4B, in that a positive pressure p₂ is applied in the second volume 43 and the resulting change of pressure p₁ in the first volume 42 is measured. In this embodiment of the bioreactor 40 (FIG. 4D) a suitably configured pressure unit 49 may be an actuator which is configured to move a part of the housing 41 of the bioreactor 40, e.g. a portion of its bottom or side wall, thus applying pressure to the culture medium 44. Alternatively, the pressure unit 49 may include a fluid reservoir in connection with a pump which is configured to provide additional culture medium 44 into the second volume 43. By applying pressure to the culture medium 44 via imposing a known volume change ΔV to the second volume 43 or the media in the second volume 44, the vertical displacement Δh of the center point of the membrane when transitioning from its relaxed state 4 into its stretched state 4* may be obtained by numerically solving equation 2 for Δh. Once Δh is known, equations 3, 3a and 3b can be applied for obtaining the elastic modulus of the (cell-covered) membrane using

Δp₁₂=Δp₁=p₁−p₀, (5)

where p₀ and p₁ refer to the pressure in the first volume 42 when the membrane is in its relaxed state 4 or its stretched state 4*, respectively.

Real-time monitoring of the elastic modulus (equation 3) requires monitoring of both p₂ and p₁ (FIG. 4B and 4C) or of both p₁ and ΔV (FIG. 4D) which can be done, but is not shown here.

Overall, it is further noted that instead of applying a positive pressure to the first volume 42 or the second volume 43, i.e. a pressure which is above ambient pressure, a negative pressure can be also applied to the first volume 42 or the second volume 43. In other words, the membrane 4 may be deformed into a stretched membrane 4* by “pulling” on it instead of “pushing” it. The terms negative and positive with respect to the pressure merely indicate the magnitude of the applied pressure relative to the ambient pressure p₀. In this context it is important to note that, the pressure p₀ refers to the pressure of the first and the second volume, when the membrane is in its relaxed state 4. Mostly for practical reasons, p₀ is referred to as ambient pressure (approximately 1 bar at sea level), but it may be set above or below the ambient atmospheric pressure. This may be achieved by statically increasing or decreasing the background pressure in the first volume 42 and/or the second volume 43. In one exemplary scenario, the whole bioreactor 40 may be placed in a pressure chamber in which the ambient or surrounding pressure p₀ may be set according to need. Increase or decrease of the ambient pressure p₀ may be particularly useful to study lung tissue at surrounding pressures different from the normal atmospheric pressure, e.g. at higher pressures which occur during diving or at lower pressures which occur during flight in an airplane.

Furthermore, even though the experimental scenarios described herein only referred to the cells being seeded and forming a confluent layer on the first side of the membrane, the membrane according to various embodiments may be also used in experimental scenarios in which cells are seeded on both sides to obtain different cell types on the two sides of the membrane. Frequently used co-culture cell models consist of epithelial cells apically (on the first surface of the membrane) and endothelial cells basally (i.e. on the second surface of the membrane). During initial growth and subsequent proliferation both cell types should remain separated to ensure that both cell types form separate confluent monolayers and do not mix during the formation of the confluent monolayers. For this process, a non-porous membrane is required, such as the membrane according to various embodiments in its configuration for the first phase. Additionally, thick electrospun membranes (thickness of 70 μm or more) have been shown to have the cells form a multi-layered cell structure not only on the surface of the membrane, but also deep within the membrane. This is also not desirable for a physiologic representation of an alveolar air-blood barrier, which consists of monolayers located at the ALI, and may be effectively avoided using the membrane according to various embodiments. An experimental configuration with cell layers on both sides of the membrane may be seen to represent an even more realistic alveolar air-blood barrier as compared to a setup comprising only one cell layer on the first surface of the membrane. Additional experimental configurations may include further cell types (e.g. macrophages, fibroblasts) and even more than two different cell types may be seeded either stacked on the first surface of the membrane and/or the second surface of the membrane or within the membrane.

The method for stretch/elasticity monitoring in real-time described herein is based on the realization that characteristic parameters of cyclic membrane stretch can be derived during cyclic membrane stretch from the pressure change in one or two air-filled volumes located in the first and/or second volume which are separated by the membrane and by using basic principles of physics. This well-known concept is applied to the specific conditions and requirements of a bioreactor such as the use of the perforated membrane according to various embodiments and contact between membrane and cell culture medium from one side (here the basal side). This approach allows for real-time monitoring of membrane elasticity, frequency of stretch and increase of membrane area during stretch by means of the rather technically simple measurement of two pressures during cell stretch.

In case the pressure is monitored only in one of the two volumes provided inside the bioreactor 40 and the displaced media volume during stretch 46 is not monitored otherwise (as described the embodiment depicted in FIG. 4D), one can only monitor the stretch parameters (amplitude and frequency), but not membrane elasticity.

It is also noted that the approach described above also works if the medium is in contact with the membrane from both sides. However, such a scenario corresponds to submerged cell culture conditions and not to the desired ALI conditions.

For the above approach to provide useful and sensible results, the membrane should be free of leaks. That is, that no gas/liquid should be allowed to pass across the membrane during the stretch cycles. For a perforated membrane with micron-sized pores as used here during the cell stretch experiments, this condition is not met by air, but is well satisfied for the aqueous cell culture medium due to its higher viscosity as a fluid. Thus, for the method disclosed herein, it is advantageous to bring the membrane in contact with liquid i.e. cell culture medium, which has a higher viscosity than gas/air and may therefore not pass through the pores of the membrane during stretch cycles. In that manner, the perforated membrane is effectively sealed during any given stretch cycle.

1. A biocompatible composite membrane for in vitro cell culturing comprising a non-water soluble, first material and a water soluble second material, wherein the composite membrane comprises a porous scaffold and a filling layer, the scaffold comprising the first material and the filling layer comprising the second material.

2. Composite membrane of claim 1,
wherein the porous scaffold is obtained by electrospinning of a substance comprising the first material; and
wherein preferably the composite membrane is obtained by spin coating of the second material onto the scaffold.

3. Method of fabricating of a biocompatible composite membrane, comprising:
providing a porous scaffold by electrospinning of a substance comprising a first material;
providing a filling layer comprising a second material on the substrate.

4. Method of claim 3, further comprising:
providing a desorption layer, preferably by spin coating, on a surface of a target onto which the substance is electrospun, the desorption layer preferably comprising the second material;
wherein preferably the desorption layer has a thickness of less than 500 nm, preferably less than 300 nm.

5. Composite membrane of claim 1, wherein the composite membrane comprises conglomerates of the second material which are embedded, as the filling layer, in the porous scaffold.

6. Composite membrane of claim 5, wherein the composite membrane is obtained by spin coating of a substance comprising the first material and the second material.

7. Composite membrane of any one of claims 1, 2, 5, 6, wherein the porous scaffold has:
a thickness of less than 10 μm, preferably less than 5 μm, most preferably less than 1 μm;
preferably a volume porosity of 1 to 50%, more preferably of 5 to 45%, even more preferably of 10 to 40%;
preferably an area- or volume-weighted median pore diameter between 0.5 and 20 μm, preferably between 1 and 15 μm, even more preferably between 2 and 10 μm;
preferably an elasticity of less than 5 MPa, more preferably less than 1 MPa, even more preferably less than 0.1 MPa.

8. Composite membrane of any one of claims 1, 2, 5-7, wherein the thickness of the filling layer is equal to or smaller than that of the fibrous scaffold, preferably less than 70% of the thickness of the porous scaffold , even more preferably less than 30% of the thickness of the porous scaffold and the duration for complete dissolution of the covering layer in water is more than 12 hours and less than 2 weeks, preferably more than 1 day and less than 1 week, even more preferably more than 1.5 days and less than 4 days.

9. Composite membrane of any one of claims 1, 2, 5-8,
wherein the first material comprises a hydrophobic material, preferably polycaprolactone;
wherein preferably the second material is wettable and preferably comprises gelatin.

10. Method of fabricating a biocompatible composite membrane for in vitro cell culturing, comprising:
spin coating of a substance comprising a first material and a second material, wherein the second material forms conglomerates which are distributed in a layer comprising the first material.

11. A bioreactor for investigating cells attached to an elastic membrane, preferably a biocompatible composite membrane of any one of the preceding claims, under stretch conditions, the bioreactor comprising:
1) a housing, having a first portion and a second portion configured to be engaged with each other and defining an inner volume;
2) the membrane, being positioned such that it divides the inner volume of the housing into a first volume, the first volume being in contact with a first surface of the membrane, and a second volume, the second volume being in contact with a second surface of the membrane;
3) a first pressure sensor for determining a pressure in the first volume;
4) a second pressure sensor for determining a pressure in the second volume; and
5) means for actively, preferably cyclically, adjusting a pressure in the first volume or the second volume.

12. Bioreactor of claim 11, wherein the means for actively adjusting a pressure in the first volume or the second volume comprises a fluid pump coupled to the respective volume via an access port.

13. Method for investigating cells attached to a membrane, preferably by means of the bioreactor according to any one of the preceding claims, comprising:
determining the elastic modulus of the membrane while actively adjusting the pressure in the first volume or in the second volume.

14. Method of claim 13, wherein investigating the cells further includes:
measuring the pressure in the respectively other volume, preferably over time while the pressure in the first or second volume is adjusted, preferably cyclically and preferably with positive differential pressure.

15. Method of claim 13 or 14, further comprising:
providing a liquid in the second volume such that the second volume is at least partly filled with the liquid and the liquid is in direct contact with the membrane.
